(19) United States
(12) Patent Application Publication
(10) Pub. No.: US 2023/0095618 A1
(43) Pub. Date: Mar. 30, 2023

(54) OIL-IN-WATER EMULSION COMPRISING A SOLID LIPID MATERIAL

(71) Applicant: Firmenich SA, Satigny (CH)

(72) Inventors: Wolfgang FIEBER, Satigny (CH); Pascal BEAUSSOUBRE, Satigny (CH); Vincent HARRACA, Satigny (CH)

(21) Appl. No.: 17/911,030

(22) PCT Filed: Mar. 29, 2021
(86) PCT No.: PCT/EP2021/058191

§ 371 Date: Sep. 12, 2022

(30) Foreign Application Priority Data
Mar. 31, 2020 (EP) 20167139.3

(51) Int. Cl. A01N 37/20; A01N 25/04; A01N 25/22; A01N 37/18; A01P 7/00

(52) U.S. Cl. CPC A01N 37/20; A01N 25/04; A01N 25/22; A01N 37/18; A01P 7/00

(57) ABSTRACT

The present invention relates to the field of delivery systems. More specifically, the invention concerns an oil-in-water emulsion comprising a solid lipid material in combination with a liquid oil-soluble active material, preferably an arthropod control agent. The present invention also describes a process for preparing said emulsion and consumer products comprising said emulsion.

### TECHNICAL FIELD

The present invention relates to the field of delivery systems. More specifically, the invention concerns an oil-in-water emulsion comprising a solid lipid material in combination with a liquid oil-soluble active material, preferably an arthropod control agent. The present invention also describes a process for preparing said emulsion and consumer products comprising said emulsion.

### BACKGROUND OF THE INVENTION

Oil-in-water emulsions are widely used in consumer products. They comprise typically a continuous aqueous phase and a dispersed oil phase containing oil-soluble active ingredients, such as for example pharmaceutical actives, emollients, essential oils, vitamins, pigments, or perfumes. A frequent usage of oil-in-water emulsions is the direct application onto skin in the form of lotions or creams. In some applications the penetration of actives through the skin is targeted. In other applications the deposition of actives on the skin is targeted. Yet in other applications the evaporation of actives from the skin is targeted.

Applications which target the evaporation of actives from the skin are for example arthropod repellents. Arthropods repelling active ingredients are mostly oil-soluble. On the one hand, they need to have a minimum vapor pressure high enough in order to yield a sufficiently high gas phase concentration to be detected by arthropods from a distance and therefore act as spatial repellent and not only as contact deterrent. On the other hand, if the vapor pressure is too high, the ingredient will have evaporated short time after having applied the lotion or the cream to the skin, therefore lacking a long-term protection against pests. In this way, most of the efficient repellent compounds present in essential oils (such as citronellal, cineol, eugenol, limonene) have a high vapor pressure and therefore can only provide a timely limited spatial repellency (Trongtokit et al. 2005:doi:10.1002/ptr.1637, Nerio et al. 2010:doi:10.1016/j.biortech.2009.07.048). Therefore, it may be desirable to provide a long-lasting release leading to a prolonged efficacy of the volatiles in the air (for example when arthropod control active ingredient is used as oil-soluble active ingredient in oil-in-water emulsions).

### SUMMARY OF THE INVENTION

The present invention solves the above-mentioned problem by providing an oil-in-water emulsion comprising a solid lipid material used in combination with a liquid oil-soluble active material.

A first object of the invention is therefore an oil-in-water emulsion comprising:
a dispersed oil phase comprising at least one solid lipid material and at least one liquid oil-soluble active ingredient, and
a continuous aqueous phase comprising a stabilizer, wherein the oil-soluble active ingredient has a Log P>0.5

A second object of the invention is a process for preparing an oil-in water emulsion, said process comprising the steps of:
(i) Dispersing an oil phase comprising at least one solid lipid material and at least one liquid oil-soluble active ingredient into a continuous aqueous phase comprising a stabilizer to obtain an oil-in-water emulsion, at a temperature above the melting point of the solid lipid material;
(ii) Cooling the emulsion thus obtained to a temperature below the melting point of the solid lipid material.

A third object of the invention is a consumer product comprising the emulsion as defined above.

A fourth object of the invention is a method for attracting or repulsing arthropod, the method comprising the steps of apply or diffusing the emulsion as defined above on a substrate or in the air.

### FIGURES

FIG. 1 represents mean number (±SD) of mosquito landings as a function of time for emulsions of the present invention compared to the pure active repellent in ethanol (Example 1).

FIG. 2 represents mean number (±SD) of mosquito landings as a function of time (n=2-4) for the emulsion of the present invention (Example 2).

FIG. 3 represents mean number (±SD) of mosquito landings as a function of time (n=2-4) for the emulsion of the present invention (Example 3).

FIG. 4 represents mean number (±SD) of mosquito landings as a function of time for emulsions of the present invention compared to the pure active repellent in ethanol (Example 4).

### DETAILED DESCRIPTION OF THE INVENTION

Unless stated otherwise, percentages (%) are meant to designate a percentage by weight of a composition.

### Oil-In-Water Emulsion

A first object of the invention is an oil-in-water emulsion comprising:
a dispersed oil phase comprising at least one solid lipid material and at least one liquid oil-soluble active ingredient, and
a continuous aqueous phase comprising a stabilizer,
wherein the oil-soluble active ingredient has a Log P>0.5

In an embodiment of the invention the emulsion comprises more than 50% water, preferably, 60%, 65%, 68%, 70%, 73%, 75%, 80%, 85% or more.

In a further embodiment of the invention the emulsion comprises more than 5% active ingredient, preferably, 10%, 12%, 15%, 20%, 25% or more.

The above preferred embodiments of the invention provide an emulsion with a high amount of water and a high amount of active ingredient relative to the other components of the emulsion. As can be appreciated, these features allow the emulsion to be prepared in a low cost and sustainable manner compared to alternative embodiments in which the more expensive, less sustainable components are at a high level.

A further embodiment of the invention is wherein the emulsion does not comprise alcohol, in particular ethanol.

Emulsion: The term “emulsion”, as used herein, denotes a mixture of two or more liquids that are normally immiscible (i.e. not soluble). In an emulsion, one liquid (the dispersed phase) is dispersed in the other (the continuous phase). The present invention is directed to oil-in water emulsions comprising a continuous hydrophilic phase in which the oil-soluble phase is dispersed. According to the invention, the oil-soluble material is in the dispersed phase. However, one part of the oil-soluble material may also be present in the continuous phase depending on the polarity of the active ingredient.

According to one embodiment, the emulsion is a macroemulsion or a nanoemulsion. An emulsion according to the invention may be prepared by any method applying mechanical forces to emulsify the disperse phase droplets, preferably by mechanical mixing with a high shear blender, a colloidal mill, an impeller mixer, or by the use of a high-pressure homogenizer. Alternatively, such emulsions may also be prepared by ultrasound processing, by membrane emulsification, or by emulsification using microfluidic channels.

The emulsion can be in the form of a gel, preferably having a viscosity comprised between 1 mPa·s and 1 Pa·s, preferably between 1 mPa·s and 500 mPa·s, wherein the viscosity is measured at 25° C. with a shear rate of 100 s⁻¹. The flow viscosity was measured using a TA Instruments AR2000 rheometer (New Castle, Del., USA) with a cone-plate geometry.

It is important to note that the emulsion of the present invention is an “oil-in-water” emulsion and not a “water-in-oil” emulsion. An “oil-in-water” emulsion has solid oil droplets in a liquid, aqueous environment, which is important for the active ingredient(s) in the emulsion. In contrast, a “water-in-oil” emulsion has water droplets in a liquid oil environment which would alter the effectiveness of the active ingredient(s) in the emulsion.

Solid lipid material: The term “solid lipid material” used in the present invention refers to lipid components that are solid or in the form of a paste at room temperature. It includes glycerides and waxes. By contrast, the term “oil” used in the present invention refers to organic components that are liquid at room temperature.

The use of a solid lipid oil material means that the active ingredient is trapped in a matrix formed by the solid lipid. Such a composition reduces the diffusivity of the active ingredient out of the matrix and slows down evaporation into the air. Hence an emulsion using a solid lipid oil material allows for a longer persistent release of the trapped active ingredient than in a liquid lipid material.

According to an embodiment, the solid lipid material is chosen in the group consisting of vegetable fats and non-vegetal fats. In one embodiment the solid fat is a derivative of vegetable fatty acids and glycerol in the form of a triglyceride. In one particular embodiment the triglyceride is palm stearin.

According to an embodiment the solid lipid material is chosen in the group consisting of vegetal waxes and non-vegetal waxes. In one particular embodiment the non-vegetal wax is beeswax. In one particular embodiment the vegetal wax is carnauba wax or jojoba wax.

According to an embodiment, the solid lipid material is chosen in the group consisting of beeswax, carnauba wax, palm stearin, jojoba wax and mixtures thereof.

According to an embodiment, the solid lipid material is not castor oil.

The amount of solid lipid material is preferably comprised between 1 to 20%, preferably between 5 to 15% by weight based on the total weight of the oil phase.

Liquid oil-soluble active material: The oil-soluble material is a single material or a mixture of materials—which forms a two-phase dispersion when mixed with water.

According to the invention, the oil-soluble active material is soluble in oil and is defined with a Log P greater than 0.5, preferably greater than 1, more preferably greater than 1.2.

According to an embodiment, the oil soluble active material has a Log P greater than 0.5 and less than 8. According to another embodiment, the oil soluble active material has a Log P greater than 0.5 and less than 5. According to another embodiment, the oil soluble active material has a Log P greater than 0.5 and less than 3.

Log P is the common logarithm of estimated octanol-water partition coefficient, which is known as a measure of lipophilicity.

The Log P values of many perfuming compound have been reported, for example, in the Pomona92 database, available from Daylight Chemical Information Systems, Inc. (Daylight CIS), Irvine, Calif., which also contains citations to the original literature. Log P values are most conveniently calculated by the “CLOGP” program, also available from Daylight CIS. This program also lists experimental log P values when they are available in the Pomona92 database. The “calculated log P” (cLog P) is determined by the fragment approach of Hansch and Leo (cf. A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each perfume oil ingredient, and takes into account the numbers and types of atoms, the atom connectivity, and chemical bonding. The cLog P values, which are the most reliable and widely used estimates for this physicochemical property, are preferably used in the selection of perfuming compounds which are useful in the present invention when no experimental Log P values are available.

According to a preferred embodiment, the ratio of active ingredient to solid lipid material is 1:1, preferably 2:1, 3:1, 4:1, 5:1, 6:1, 7:1, 8:1, 9:1, 10:1 or more. The experimental data below provides examples of favourable active ingredient to solid lipid material ratios.

According to a preferred embodiment, the active ingredient comprises at least one arthropod control agent.

As shown in the accompanying examples, when the emulsion of the invention comprises at least one arthropod control agent, the agent impart a long lasting arthropod control activity to the emulsion. While the term “long lasting” is subjective, in this context it is intended to mean that the arthropod control activity of the arthropod control agent persists for a longer time when prepared in the form of the emulsion of the invention than in an alternative form where the arthropod control agent is not present in the emulsion of the invention. Though not being intended to limited the scope of the invention, it is the opinion of the inventors of the present invention that the arthropod control agent is present in a stablised form in the emulsion and is released in a slower manner to provide a longer lasting repellency than other formulations in which the arthropod control agent is prepared or in a free form, i.e. not in any emulsion at all.

The term “arthropod” has the normal meaning for a skilled person in the technical field. Arthropods include invertebrate animals, such as insects, arachnids, and crustaceans, that have a segmented body and jointed appendages. Arthropods usually have a chitinous exoskeleton molted at intervals, and a dorsal anterior brain connected to a ventral chain of ganglia.

Arthropods in the present invention's understanding relate to undesired arthropods, meaning that their presence in the air, on the surface of an article, the surface of a plant or the surface of a vertebrate, such as a human subject or other mammal, preferably human subject, is not desired. Preferably undesired arthropods are pest arthropods that impact plants and animals, e.g. thrips, aphids, beetles, moth, mealybug, scale etc., more preferably pest arthropods that impact animals, e.g. ants, termites, cockroaches, flies, etc., even more preferably blood feeding arthropods that impact vertebrates, e.g. biting fly, bed bug, kissing bug, flea, lice, mosquitos and ticks, even more preferably mosquitos and ticks.

The reason why the presence of an arthropod is not desired might be that the arthropod's presence in the air is unpleasant to a subject, the contact of an arthropod on an article transfers diseases and/or germs or the arthropod bites an organism and causes itching, the transmission of diseases and/or germs or the arthropod feeding may be the cause for other diseases and/or conditions.

The expression “control”, “arthropod control” or the like has the normal meaning for a skilled person in the technical field. “Controlling” in the context of the present invention defines the ability of a compound or an arthropod controlling composition according to the present invention to attract, deter, kill or repel an arthropod, preferably deter or repel an arthropod and even more preferably repel an arthropod.

“Attracting” according to the present invention defines the ability of a compound or an arthropod attractant composition according to the invention to increase or encourage contact or the presence of an arthropod at the arthropod attractant source, such as in the air, on the surface of an article or on the surface of an vertebrate, such as a human subject or other mammal, preferably an article such as a trapping device, the arthropod attractant compound or composition has been applied to.

“Deterring” according to the present invention defines the ability of a compound or an arthropod deterrent composition according to the invention to minimize, reduce, discourage or prevent contact or the presence of an arthropod at the arthropod deterrent source, such as in the air, on the surface of an article or on the surface of an vertebrate, such as a human subject or other mammal, preferably human subject, to which the arthropod deterrent compound or composition has been applied to. Typically, the deterrent effect is shown when used as feeding deterrent hindering a pest from subsequent food intake or oviposition after an initial tasting of the arthropod deterrent compound or composition.

“Killing” according to the present invention defines the ability of a compound or an art report killing composition according to the present invention to kill an arthropod at the arthropod killing source, such as in the air, on the surface of an article or on the surface of an vertebrate, such as a human subject or other mammal, preferably human subject, to which the arthropod killing compound or composition has been applied to. When an arthropod killing composition is applied to a plant, an animal or human subject, it is applied in an amount which is killing to the arthropod but not to the subject.

“Repellency” according to the present invention defines the ability of a compound or an arthropod repellent composition according to the present invention to minimize, reduce, discourage or prevent approach or the presence of an arthropod at the arthropod repellent source, such as in the air, on the surface of an article or on the surface of an vertebrate, such as a human subject or other mammal, preferably human subject, to which the arthropod repellent compound or composition has been applied to.

Alternative ingredients could be used either instead of an arthropod control agent, or in combination with an arthropod control agent. Non-limiting examples of such ingredients include a perfume, cosmetic, skin caring, malodour counteracting, bactericide, fungicide, pharmaceutical or agrochemical ingredient, a microbial agent, a sanitizing agent, and mixture thereof.

According to a particular embodiment, the arthropod control agent is used in combination with a perfume, with optional substances which together improve, enhance or modify the delivery of the perfuming ingredients, such as perfume precursors, emulsions or dispersions, as well as combinations which impart an additional benefit beyond that of modifying or imparting an odor, such as long-lasting, blooming, malodor counteraction, antimicrobial effect or microbial stability.

According to an embodiment, when the arthropod control agent is used in combination with a perfume, the arthropod control agent is used preferably in an amount comprised between 5 and 40%, more preferably between 10 and 30% based on the total composition of the emulsion, and the perfume is used preferably in an amount comprised between 0.1 to 5%, more preferably between 0.5 and 2% based on the total composition of the emulsion.

According to a particular embodiment, the arthropod control agent is a perfuming ingredient.

The nature and type of the arthropod control agent that can be present in the oil-soluble internal phase do not warrant a more detailed description here, which in any case would not be exhaustive, the skilled person being able to select them on the basis of its general knowledge and according to the intended use or application.

According to an embodiment, the oil-soluble active ingredient is an arthropod control ingredient or a mixture of arthropod control ingredients.

According to an embodiment, the oil-soluble active ingredient is an not UV-blocking agent.

According to a particular embodiment, the arthropod control ingredient is chosen in the group consisting of ethyl 3-(acetyl(butyl)amino)propanoate (IR3535®, N,N-diethyl-3-methylbenzamide (DEET), p-menthane-3,8-diol (PMD), Eucalyptus citriodora oil, Citronella spp. oil, sec-butyl 2-(2-hydroxyethyl)piperidine-1-carboxylate (picaridin), vanillin, Castor oil, Cedarwood oil, Cinnamon oil, Citronella, Citronella oil, Clove oil, Corn oil, Cornmint, Cornmint oil, Cottonseed oil, 4-Allyl-2-methoxyphenol (Eugenol), Garlic oil, (2E)-3,7-Dimethylocta-2,6-dien-1-ol (Geraniol), Geranium oil, Lemongrass oil, Linseed oil, Peppermint, Peppermint oil, 2-Phenylethyl propionate, Rosemary oil, Sesame oil, Soybean oil, Spearmint, Spearmint oil, Thyme oil, Mint, Mint oil, Pepper extract, Wintergreen oil, citronellal, Lavender oil, Lavandula hybrida ext., Lavandin oil, Lemon oil, Margosa extract, Mentha arvensis ext., Metofluthrin, Nonanoic acid, Pyrethrins and Pyrethroids, 2,3,4,5-bis(butyl-2-ene)tetrahydrofurfural (MGK Repellent 11), cineole, cinnamaldehyde, citral, citronellol, Citronella oil, coumarin, dibutyl phthalate, diethyl phthalate, dimethyl anthranilate, dimethyl phthalate, ethyl vanillin, eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, lime oil, limonene, linalool, methyl anthranilate, Mint arvensis, Mint spicata, Myrcene, Neem oil, Sabinene, β-Caryophyllene, (1H-indol-2-yl)acetic acid, anethole, anise oil, basil oil, bay oil, camphor, ethyl salicylate, evergreen oils, pine oil, Tetramethrin, Allethrin, (RS)-α-cyano-3phenoxybenzyl-(1RS)-cis, Cypermethrin, Prallethrin, Acetamiprid, Azadirachtin, Bendiocarb, Bifenthrin, Chlorpyrifos, deltamethrin, Diazinon, Dichlorvos, fipronil, imidacloprid, Linalool, Malathion, Margosa extract, Nicotine, Permethrin, rotenone, S-Methoprene, Spinosad (Spinosyn A), Spinosyn D, Transfluthrin, anisic alcohol, octahydrocoumarin, (++2,5-dimethyl-2-indanmethanol, 4,4A,5,9B-tetrahydroindeno[1,2-D]-1,3-dioxin, 2,4-dimethyl-4,4a,5,9b-tetrahydroindeno[1,2-d][1,3]dioxine, and mixtures thereof.

A preferred embodiment of the invention is wherein the arthropod control ingredient is not N,N-diethyl-3-methylbenzamide (DEET).

A preferred embodiment of the invention is wherein the arthropod control ingredient is not ethyl 3-(acetyl(butyl)amino)propanoate)(IR3535°.

In addition to the oil-soluble active material, the continuous phase can comprise a hydrophilic active ingredient dispersed or solubilized, preferably chosen in the group consisting of dried blood, lauryl sulfate, malic acid, Potassium (2E,4E)-hexa-2,4-dienoate, putrescent whole egg solids, sodium chloride, sulfuric acid monododecyl ester, sodium salt, zinc, boric acid, citric acid, maltodextrin, silicium dioxide, and mixtures thereof.

By “perfume oil” (or also “perfume”) what is meant here is an ingredient or composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lasting, blooming, malodour counteraction, antimicrobial effect, microbial stability, arthropods control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said ingredients may also be compounds known to release in a controlled manner various types of perfuming compounds.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very oil-soluble and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

According to an embodiment, the emulsion can comprise a co-solvent. According to an embodiment, the co-solvent is a vegetable oil and/or an animal oil.

Oil-miscible co-solvent: According to an embodiment, the dispersed phase comprises an oil-miscible co-solvent.

An oil-miscible co-solvent which can be used in the invention may be, for example, tributyl-0-acetylcitrate, triethylcitrate, caprylic triglyceride, triacetin, coconut alkanes (and) coco-caprylate/caprate, propanediol dicaprylate, octanoic acid 1,3-propanediyl ester, isopropyl palmitate, isopropyl myristate, ethyl oleate, triheptanoin, caprylic/capric glycerides, undecane and tridecane, C15-C19 alkanes, squalene, a silicone oil, a glycol ether such as tripropylene glycol methyl ether, dipropylene glycol n-propyl ether, DIPG monomethyl ether, a dimethyl adipate/dimethyl glutarate ester, benzyl benzoate, piperonil butoxide, coconut oil, or mixtures thereof.

Preferably the oil-miscible co-solvent chosen in the group consisting of benzyl benzoate, piperonil butoxide, coconut oil and mixtures thereof in order to get a homogeneous dispersed phase. When present, the co-solvent is preferably used between 5 and 30%, preferably between 10 and 25% by weight based on the total weight of the oil phase.

Water-miscible co-solvent: According to an embodiment, the aqueous continuous phase comprises a water-miscible co-solvent, preferably chosen in the group consisting of mono- and polyhydric solvents. Non limiting examples of such solvents can be found from the group containing ethanol, propanol, propylene glycol, hexylene glycol, dipropylene glycol, glycerol, isopropylidene glycerol, butylene glycol (1,3-butanediol) and isopropanol, and mixtures thereof. When present, the co-solvent is preferably used between 5 and 30%, preferably between 10 and 25% by weight based on the total weight of the aqueous phase.
Stabilizer: According to the invention, the continuous phase comprises a stabilizer dispersed in water.
According to a particular embodiment, the stabilizer is a molecular emulsifier.
“Molecular emulsifier” are amphiphilic molecules that concentrate at the interface between two phases and modify the properties of that interface. Examples of stabilizers can be found in McCutcheon's Stabilizers & Detergents or the Industrial Surfactants Handbook.
According to an embodiment, the molecular emulsifier is a polymeric emulsifiers.

According to an embodiment, the molecular emulsifier is not a non-ionic emulsifiers.

The use polymeric emulsifiers is preferred since these stabilizing agents have a higher stability (i.e. charge repulsion) than non-ionic emulsifiers and also prevents aggregation between the solid droplets.

Also, non-ionic emulsifiers such as PEG and PPG-based surfactants are non-natural and therefor by excluding this class of molecule from the emulsion of the invention there is no risk of contaminants such as 1,4 dioxane, free glycol ether and free ethylene oxides.

An additional advantage of polymeric emulsifiers is that such molecules are List 25b compliant in the context of US EPA approval meaning that there are less regulatory obstacles for the commercial exploitation of the emulsion of the invention.

As non-limiting examples, the molecular emulsifier can be chosen in the group consisting of modified starch, Gum Arabic, Pectins, Casein, cyclodextrins, lecithins, soy protein, quillaja saponin, and mixtures thereof.

When present, the stabilizer is preferably used between 0.5% and 15%, preferably between 1 and 10% by weight based on the total weight of the emulsion.

Optional ingredients: The emulsion may also comprise optional ingredients such as a weighting agent (such as Damar gum, acetyl tributyl citrate), a viscosifier, a gelling agent (such as Agar gum, gellan gum, guar gum, tragacanth gum, cellulose derivatives, xanthan gum), pH adjusters, and mixtures thereof.
Examples of Viscosifiers which can be Used in the Present Invention Include Carboxylic Acid Homopolymer, Carboxylic Acid Copolymers

According to the present invention, an emulsion composition may further comprise optional other ingredients such as colorants, preservatives, emollients, humectants, antioxidants, free radical scavengers, POV remediation agents, cooling agents, vitamins, fixatives, cosmetic benefit agents, chelators, functional polymers, or electrolytes.

Such optional ingredients may represent no more than 10%, 3% w/w, or even 2% w/w, the percentages being relative to the total weight of the emulsion.

Examples of cooling agents which can be used in the present invention include menthol, menthol methyl ether, menthol ethylene glycol carbonate (FEMA GRAS 3805), menthol propylene glycol carbonate (FEMA GRAS 3806), menthyl-N-ethyloxamate, monomenthyl succinate (FEMA GRAS 3810), monomenthyl glutamate (FEMA GRAS 4006), menthoxy-1,2-propanediol (FEMA GRAS 3784), 3-hydroxymethyl p-menthane, menthyl ethoxyhydroxyacetate, 2-(4-ethylphenoxy)-N-(1H-pyrazol-5-yl)-N-(2-thienylmethyl)acetamide, WS23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (1R,2S,5R)—N-(4-Methoxyphenyl)-p-menthanecarboxamide, FEMA 4681; WS27 (N-Ethyl-2,2-diisopropylbutanamide), FEMA 4557; N-Cyclopropyl-5-methyl-2-isopropylcyclohexanecarboxamide, FEMA 4693, WS-116 (N-(1,1-Dimethyl-2-hydroxyethyl)-2,2-diethylbutanamide), N-(1,1-Dimethyl-2-hydroxyethyl)2,2-diethylbutanamide, FEMA 4603, Menthoxyethanol, FEMA 4154, N-(4-cyanomethylphenyl)-p-menthanecarboxamide, FEMA 4496; N-(2-(Pyridin-2-yl)ethyl)-3-p-menthanecarboxamide, FEMA 4549; N-(2-Hydroxyethyl)-2-isopropyl-2,3-dimethylbutanamide, FEMA 4602 and (also N-(4-(carbamoylmethyl)phenyl)-menthylcarboxamide, FEMA 4684; (1R,2S,5R)—N-(4-Methoxyphenyl)-p-menthanecarboxamide (WS-12), FEMA 4681; (2S,5R)—N-[4-(2-Amino-2-oxoethyl)phenyl]-p-menthanecarboxamide, FEMA 4684; and N-Cyclopropyl-5-methyl-2-isopropylcyclohexanecarbonecarboxamide, FEMA 4693; 2-[(2-p-Menthoxy)ethoxy]ethanol, FEMA 4718; (2,6-Diethyl-5-isopropyl-2-methyltetrahydropyran, FEMA 4680); trans-4-tert butyl cyclohexanol, FEMA 4724; 2-(p-tolyloxy)-N-(1H-pyrazol-5-yl)-N-((thiophen-2-yl)methyl)acetamide, FEMA 4809; Menthone glycerol ketal, FEMA 3807; Menthone glycerol ketal, FEMA 3808; (−)-Menthoxypropane-1,2-diol; 3-(L-Menthoxy)-2-methylpropane-1,2-diol, FEMA 3849; isopulegol; (+)-cis & (−)-trans p-Menthane-3,8-diol, Ratio ˜62:38, FEMA 4053; 2,3-dihydroxy-p-menthane; 3,3,5-trimethylcyclohexanone glycerol ketal; menthyl pyrrolidone carboxylate; (1R,3R,4S)-3-menthyl-3,6-dioxa heptanoate; (1R,2S,5R)-3-menthyl methoxyacetate; (1R,2S,5R)-3-menthyl 3,6,9-trioxadecanoate; (1R,2S,5R)-3-menthyl 3,6,9-trioxadecanoate; (1R,2S,5R)-3-menthyl (2-hydroxyethoxy)acetate; (1R,2S,5R)-menthyl-11-hydroxy-3,6,9-trioxaundecanoate; Cubebol, FEMA 4497; N-(4-cyanomethylphenyl) p-menthanecarboxamide, FEMA 4496; 2-isopropyl-5-methylcyclohexyl 4-(dimethylamino)-4-oxobutanoate, FEMA 4230; N-(4-cyanomethylphenyl) p-menthanecarboxamide, FEMA 4496; N-(2-pyridin-2-ylethyl) p-menthanecarboxamide, FEMA 4549, Menthyl lactate, FEMA 3748; 6-isopropyl-3,9-dimethyl-1,4-dioxaspiro[4.5]decan-2-one, FEMA 4285; N-benzo[1,3] dioxol-5-yl-3-p-menthanecarboxamide; N-(1-isopropyl-1,2-dimethylpropyl)-1,3-benzodioxole-5-carboxamide; N-(R)-2-oxotetra hydrofuran-3-yl-(1R,2S,5R)-p-menthane carboxamide; mixture of 2,2,5,6,6-pentamethyl-2,3,6,6a-tetrahydropentalen-3a(1H)-ol and 5-(2-hydroxy-2-methylpropyl)-3,4,4-trimethylcyclopent-2-en-1-one; (1R,2S,5R)-2-isopropyl-5-methyl-N-(2-(pyridin-2-yl)ethyl)cyclohexanecarboxamide, FEMA 4549; (2S,5R)-2-isopropyl-5-methyl-N-(2-(pyridin-4-yl)ethyl)cyclohexanecarboxamide; N-(4-cyanomethylphenyl) p-menthanecarboxamide, FEMA 4496; (1S,2S,5R)—N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-yl)ethyl]benzenesulfonamide; 4-cyano-N-phenyl-N-[2-(pyridin-2-yl)ethyl] benzenesulfonamid; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol, 4-((9H-fluoren-9-ylamino)methyl)-2-methoxyphenol; 4-((benzhydrylamino)methyl)-2-ethoxyphenol; 1-(4-methoxyphenyl)-2-(1-methyl-1H-benzo[d]imidazol-2-yl)vinyl4-methoxybenzoate; 2-(1-isopropyl-6-methyl-1H-benzo[d]imidazol-2-yl)-1-(4-methoxyphenyl)vinyl4-methoxybenzoate; (Z)-2-(1-isopropyl-5-methyl-1H-benzo[d]imidazol-2-yl)-1-(4-methoxy-phenyl)vinyl-4-methoxybenzoate; 3-alkyl-p-menthan-3-ol derivatives; derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl; menthyl oxamate derivatives; menthyl 3-oxocarboxylic acid esters; N alpha-(Menthanecarbonyl)amino acid amides; p-menthane carboxamide and WS-23 analogs; (−)-(1R,2R,4S)-dihydro-umbellulol; p-menthane alkyloxy amides; cyclohexane derivatives; butone derivatives; a mixture of 3-menthoxy-1-propanol and 1-menthoxy-2-propanol; 1-[2-hydroxyphenyl]-4-[2-nitrophenyl-]-1,2,3,6-tetrahydropyrimidine-2-one; 4-methyl-3-(1-pyrrolidinyl)-2[5H]-furanone; and combinations thereof.

Examples of fixatives which can be used in the present invention include, for example, caprylyl alcohol, octanol, butyloctanol, isotridecyl alcohol, hexyldecanol, isocetyl alcohol, isostearyl alcohol, octyldecanol, octyldodecanol, decyltetradecanol, tetradecyloctadecanol, neopentyl glycol diethylhexanoate, PPG-3 myristyl ether, and PPG-20 methyl glucose ether.

According to an embodiment, the emulsion does not contain a sun-block agent, and the emulsion does not function as a sun block.

### Process for Preparing the Oil-In-Water Emulsion

Another object of the invention is a process for preparing an oil-in water emulsion, said process comprising the steps of:
(i) Dispersing an oil phase comprising at least one solid lipid material and at least one liquid oil-soluble active ingredient into a continuous aqueous phase comprising a stabilizer to obtain an oil-in-water emulsion, at a temperature above the melting point of the solid lipid material;
(ii) Cooling the emulsion thus obtained to a temperature below the melting point of the solid lipid material.

According to an embodiment, the melting temperature of the dispersed phase is comprised between 40° C. and 80° C., preferably between 40° C. and 70° C., more preferably 45° C. and 65° C.

Hence an embodiment of the invention is wherein step i) is performed at less than 65° C. An advantage of this embodiment of the invention is there will be less degradation of the active ingredient than at a temperature above 65° C. Moreover at a lower temperature more of the active ingredient will be contained within the solid material matrix and hence not exposed to water or air during storage of the emulsion before use.

In step i), stabilizer(s) and ingredients forming the aqueous phase are mixed with water to obtain a homogeneous continuous phase. The lipid mixture (solid lipid material and liquid oil-soluble material) is added to the continuous phase at room temperature and the ingredients are heated up together above the melting point of the solid lipid material prior to emulsification. Alternatively, the lipid mixture can also be heated separately above the melting point of the solid lipid material and then added to the warm water phase prior or during the emulsification step. The emulsification step consists of using any known emulsifying method, such as high shear mechanical mixing, sonication or high-pressure homogenization. Such emulsifying methods are well known to the person skilled in the art.

Advantageously, the emulsion presents a drop size having an average diameter (d₅₀) of between 0.1 to 20 microns, preferably 0.5 to 20 microns, most preferably 0.5 to 10 microns.

The drop size can be measured via any well-established method that allows measurements which are accurate within an experimental error of 5% at the most and preferably below 1%. Suitable well-established methods use laser diffraction particle size analyser (e.g. Coulter LS 13 320 from Beckman Coulter, Brea, Calif., USA). Upon analysis the volume statistics (d_{4,3}) was determined to characterize the emulsion.

In step ii) of the process, the emulsion thus obtained is cooled to a temperature below the melting point of the dispersed phase.

Typically, the cooling step is done by reducing temperature of the emulsion of 5 to 30° C. per hour, preferably of 10 to 25° C. per hour, preferably of 12 to 15° C. per hour.

### Consumer Products

The invention's emulsions can advantageously be used in different fields, such as perfumery industry.

Consequently, another object of the present invention is represented by a consumer product comprising the emulsion as defined above.

The nature and type of the constituents of the consumer product do not warrant a more detailed description here, which in any case would not be exhaustive, the skilled person being able to select them on the basis of his general knowledge and according to the nature and the desired effect of said product. These formulations do not warrant a detailed description here which would in any case not be exhaustive. The person skilled in the art of formulating such consumer products is perfectly able to select the suitable components on the basis of his general knowledge and of the available literature.

According to a particular embodiment, the consumer product is a perfuming consumer product.

For the sake of clarity, it has to be mentioned that, by “perfuming consumer product” it is meant a consumer product which is expected to deliver at least a pleasant perfuming effect to the surface to which it is applied (e.g. skin, hair, textile, or home surface). In other words, a perfuming consumer product according to the invention is a perfumed consumer product which comprises a functional formulation, as well as optionally additional benefit agents, corresponding to the desired consumer product, e.g. a detergent or an air freshener, or the delivering of a technical benefit such as an arthropod control ingredient.

Non-limiting examples of suitable perfumery consumer product can be a perfume, such as a fine perfume, a body splash, a cologne or an after-shave lotion; an arthropod repellent lotion, a fabric care product, such as a liquid detergent, a fabric softener, a fabric refresher, an ironing water, or a bleach; a body-care product, such as a hair care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener; or a home care product, such all-purpose cleaners, liquid dishwashing products, toilet cleaners or products for cleaning various surfaces, for example sprays & wipes intended for the treatment/refreshment of textiles or hard surfaces (floors, tiles, stone-floors etc.).

According to a particular embodiment, the oil-soluble active ingredient comprises an arthropod control ingredient and the consumer product is an arthropod control article.

By “arthropod control article” is understood to designate a consumer product which delivers at least an arthropod controlling effect to the surface or space to which it is applied (e.g. skin, hair, textile, or home surface). In other words, an arthropod controlling article according to the invention is a consumer product which comprises the emulsion of the invention, as well as optionally additional benefit agents, corresponding to the desired consumer product, and an arthropod controlling amount of at least arthropod control ingredient contained in the emulsion. For the sake of clarity, said consumer product is a non-edible product.

The consumer product can be in the form of a sprayable solution, or a gel/viscous lotion (roll-on or sticks).

The invention will now be further described by way of examples. It will be appreciated that the invention as claimed is not intended to be limited in any way by these examples.

### EXAMPLES

### Example 1

### Emulsions According to the Invention (with IR3535®)

In a 50 ml glass flask, 0.04 g of sodium benzoate, 0.04 g of potassium sorbate, 0.70 g of sodium citrate monobasic, 1.51 g of sodium citrate dibasic sesquihydrate and 5 g of gum arabic were added to 42.70 g of water. The water phase was let for 4 hrs under magnetic stirring to solubilize the ingredients and get a clear and homogeneous phase.
In a 20 ml glass vial, emulsion 1 was prepared by weighing 1.25 g of IR3535®, 0.15 g of Beeswax, 0.3 g of resinogum Damar and 0.3 g of benzyl benzoate. The oil phase was heated up to 70° C. for 30 minutes to get a liquid and homogenous oil phase. Separately, water phase was heated up to 70° C. and 8 g were added to the oil phase. The warm solution containing the 2 phases was sheared for 2 minutes using an ultrasonic probe to produce an oil-in-water emulsion. The sample was then immersed in a water bath and left to cool down to room temperature.

In another 20 ml glass vial, emulsion 2 was prepared by weighing 1.25 g of IR3535®, 0.15 g of Beeswax and 0.4 g of Tributyl-O-acetylcitrate. The oil phase was heated up to 70° C. for 10 minutes to get a liquid and homogenous oil phase. Separately, water phase was heated up to 70° C. and 8.2 g were added to the oil phase. The warm solution containing the 2 phases was sheared for 2 minutes using an ultrasonic probe to produce an oil-in-water emulsion. The sample was then immersed in a water bath and left to cool down to room temperature.

TABLE 1
Emulsion compositions

| | Emulsion 1 | Emulsion 2 | |
|---|---|---|---|
| | Effective Amount of | | |
| Ingredients | Ingredient (% wt)^{a)} | | Function |
| Gum arabic ¹⁾ | 8.00 | 8.20 | Stabilizer |
| Sodium citrate dibasic | 2.41 | 2.47 | pH adjuster |
| sesquihydrate ²⁾ | | | |
| Sodium citrate | 1.12 | 1.15 | pH adjuster |
| monobasic ³⁾ | | | |
| Sodium benzoate ⁴⁾ | 0.07 | 0.07 | Preservative |
| | | | agent |
| Potassium sorbate ⁵⁾ | 0.07 | 0.07 | Preservative |
| | | | agent |
| Demineralized water | 68.32 | 70.03 | Solvent |
| IR3535 ® ⁶⁾ | 12.5 | 12.5 | Arthropod |
| | | | control agent |
| Beeswax ⁷⁾ | 1.5 | 1.5 | Solid lipid |
| | | | material |
| Resinogum Damar ⁸⁾ | 3 | | Weighting agent |
| Tributyl-O- | | 4 | Weighting agent |
| acetylcitrate ⁹⁾ | | | |
| Benzyl benzoate ¹⁰⁾ | 3 | | Oil-miscible |
| | | | co-solvent |

¹⁾ Superstab ™ gum; origin: Nexira
²⁾ Origin: Sigma Aldrich
³⁾ Origin: Sigma Aldrich
⁴⁾ Origin: Alfa Aesar
⁵⁾ Origin: Alfa Aesar
⁶⁾ Ethylbutylacetylamino propionate; Origin: Merck
⁷⁾ Origin: Aldrich
⁸⁾ Damar-EZ ™; Origin: Nexira
⁹⁾ Origin: Sigma Aldrich
¹⁰⁾ Origin: Firmenich

Homogeneous and sprayable emulsions were obtained.

### Example 2

### Emulsion According to the Invention (with IR3535®)

In a 20 ml glass vial, 0.007 g of sodium benzoate and 0.007 g of potassium sorbate were added to 7.0 g of a pH5 buffered water solution (citrate buffer). Once a clear solution was obtained, 0.78 g of gum arabic was added under mechanical stirring to get a homogeneous water solution. Then 1.25 g of IR3535®, 0.15 g of Beeswax, 0.3 g of resinogum Da mar and 0.3 g of benzyl benzoate were added to the water phase. The sample was heated up to 65° C. to melt the Beeswax. An amount of 0.2 g of fragrance was added and the solution was sheared for 1 minute using an ultrasonic probe to produce an oil-in-water emulsion. The sample was then immersed in a water bath and left to cool down to room temperature.

TABLE 2
Emulsion composition

| | Effective Amount of | |
|---|---|---|
| Ingredients | Ingredient (% wt)^{a)} | Function |
| Gum arabic ¹⁾ | 7.80 | Stabilizer |
| Sodium citrate dibasic | 2.38 | pH adjuster |
| sesquihydrate ²⁾ | | |
| Sodium citrate | 1.11 | pH adjuster |
| monobasic ³⁾ | | |
| Sodium benzoate ⁴⁾ | 0.07 | Preservative |
| | | agent |
| Potassium sorbate ⁵⁾ | 0.07 | Preservative |
| | | agent |
| Demineralised water | 66.57 | Solvent |
| IR3535 ® ⁶⁾ | 12.5 | Arthropod |
| | | control agent |
| Fragrance ⁷⁾ | 2.0 | Fragrance |
| Beeswax ⁸⁾ | 1.5 | Solid lipid |
| | | material (matrix |
| | | former) |
| Resinogum Damar ⁹⁾ | 3 | Weighting agent |
| Benzyl benzoate ¹⁰⁾ | 3 | Oil-miscible |
| | | co-solvent |

¹⁾ Superstab ™ gum; origin: Nexira
²⁾ Origin: Sigma Aldrich
³⁾ Origin: Sigma Aldrich
⁴⁾ Origin: Alfa Aesar
⁵⁾ Origin: Alfa Aesar
⁶⁾ Ethylbutylacetylamino propionate; Origin: Merck
⁷⁾ Origin: Firmenich
⁸⁾ Origin: Aldrich
⁹⁾ Damar-EZ ™; Origin: Nexira
¹⁰⁾ Origin: Firmenich

An homogeneous and sprayable emulsion was obtained.

### Example 3

### Emulsion According to the Invention (with IR3535®)

In a 20 ml glass vial, 0.007 g of sodium benzoate and 0.007 g of potassium sorbate were added to 7.5 g of a pH5 buffered water solution (citrate buffer). Once a clear solution was obtained, 0.3 g of pectin was added at 40° C. under mechanical stirring to get and homogeneous water solution. Then 1.25 g of IR3535®, 0.15 g of Beeswax, 0.3 g of resinogum Damar and 0.3 g of benzyl benzoate were added to the water phase. The sample was heated up to 65° C. to melt the Beeswax. An amount of 0.1 g of fragrance was added and the solution was sheared for 1 minute using an ultrasonic probe to produce an oil-in-water emulsion. The sample was then immersed in a water bath and left to cool down to room temperature.

TABLE 3
Emulsion composition

| | Effective Amount of | |
|---|---|---|
| Ingredients | Ingredient (% wt)^{a)} | Function |
| Pectin ¹⁾ | 3.00 | Stabilizer |
| Sodium citrate dibasic | 2.50 | pH adjuster |
| sesquihydrate ²⁾ | | |
| Sodium citrate | 1.17 | pH adjuster |
| monobasic ³⁾ | | |
| Sodium benzoate ⁴⁾ | 0.08 | Preservative |
| | | agent |
| Potassium sorbate ⁵⁾ | 0.08 | Preservative |
| | | agent |
| Demineralised water | 71.17 | Solvent |
| IR3535 ® ⁶⁾ | 12.5 | Arthropod |
| | | control agent |
| Fragrance ⁷⁾ | 2.0 | Fragrance |
| Beeswax ⁸⁾ | 1.5 | Solid lipid |
| | | material |
| Resinogum Damar ⁹⁾ | 3 | Weighting agent |
| Benzyl benzoate ¹⁰⁾ | 3 | Oil-soluble |
| | | co-solvent |

¹⁾ GENU ®Beta Pectin; origin: CP Kelco
²⁾ Origin: Sigma Aldrich
³⁾ Origin: Sigma Aldrich
⁴⁾ Origin: Alfa Aesar
⁵⁾ Origin: Alfa Aesar
⁶⁾ Ethylbutylacetylamino propionate; Origin: Merck
⁷⁾ Origin: Firmenich
⁸⁾ Origin: Aldrich
⁹⁾ Damar-EZ ™; Origin: Nexira
¹⁰⁾ Origin: Firmenich

An homogeneous and sprayable emulsion was obtained.

### Example 4

### Emulsions According to the Invention (with DEET)

In a 50 ml glass flask, 0.04 g of sodium benzoate, 0.04 g of potassium sorbate, 0.70 g of sodium citrate monobasic, 1.51 g of sodium citrate dibasic sesquihydrate and 5 g of gum arabic were added to 42.70 g of water. The water phase was let for 4 hrs under magnetic stirring to solubilize the ingredients and get a clear and homogeneous phase.
In a 20 ml glass vial, emulsion 1 was prepared by weighing 1.25 g of DEET and 0.15 g of Beeswax. The oil phase was heated up to 70° C. for 10 minutes to get a liquid and homogenous oil phase.
Separately, water phase was heated up to 70° C. and 8.6 g were added to the oil phase. The warm solution containing the 2 phases was sheared for 2 minutes using an ultrasonic probe to produce an oil-in-water emulsion. The sample was then immersed in a water bath and left to cool down to room temperature.
In another 20 ml glass vial, emulsion 2 was prepared by weighing 1.25 g of DEET, 0.25 g of Beeswax and 0.3 g of resinogum Damar. The oil phase was heated up to 70° C. for 30 minutes to get a liquid and homogenous oil phase. Separately, water phase was heated up to 70° C. and 8.2 g were added to the oil phase. The warm solution containing the 2 phases was sheared for 2 minutes using an ultrasonic probe to produce an oil-in-water emulsion. The sample was then immersed in a water bath and left to cool down to room temperature.
In another 20 ml glass vial, emulsion 3 was prepared by weighing 1.25 g of DEET, 0.15 g of Beeswax and 0.4 g of Tributyl-O-acetylcitrate. The oil phase was heated up to 70° C. for 10 minutes to get a liquid and homogenous oil phase. Separately, water phase was heated up to 70° C. and 8.2 g were added to the oil phase. The warm solution containing the 2 phases was sheared for 2 minutes using an ultrasonic probe to produce an oil-in-water emulsion. The sample was then immersed in a water bath and left to cool down to room temperature.

TABLE 4
Emulsion compositions

| | Emul- | Emul- | Emul- | |
|---|---|---|---|---|
| | sion 1 | sion 2 | sion 3 | |
| | Effective Amount of | | | |
| Ingredients | Ingredient (% wt)^{a)} | | | Function |
| Gum arabic ¹⁾ | 8.60 | 8.20 | 8.20 | Stabilizer |
| Sodium citrate dibasic | 2.59 | 2.47 | 2.47 | pH adjuster |
| sesquihydrate ²⁾ | | | | |
| Sodium citrate | 1.21 | 1.15 | 1.15 | pH adjuster |
| monobasic ³⁾ | | | | |
| Sodium benzoate ⁴⁾ | 0.08 | 0.07 | 0.07 | Preservative |
| | | | | agent |
| Potassium sorbate ⁵⁾ | 0.08 | 0.07 | 0.07 | Preservative |
| | | | | agent |
| Demineralized water | 73.45 | 70.03 | 70.03 | Solvent |
| DEET ⁶⁾ | 12.5 | 12.5 | 12.5 | Arthropod |
| | | | | control agent |
| Beeswax ⁷⁾ | 1.5 | 2.5 | 1.5 | Solid lipid |
| | | | | material |
| Resinogum Damar ⁸⁾ | | 3 | | Weighting |
| | | | | agent |
| Tributyl-O- | | | 4 | Weighting |
| acetylcitrate ⁹⁾ | | | | agent |

¹⁾ Superstab ™ gum; origin: Nexira
²⁾ Origin: Sigma Aldrich
³⁾ Origin: Sigma Aldrich
⁴⁾ Origin: Alfa Aesar
⁵⁾ Origin: Alfa Aesar
⁶⁾ N,N-diethyl-3-methylbenzamide; Origin: Sigma Aldrich
⁷⁾ Origin: Aldrich
⁸⁾ Damar-EZ ™; Origin: Nexira
⁹⁾ Origin: Sigma Aldrich

Homogeneous and sprayable emulsions were obtained.

### Example 5

### Repulsive Effect on Mosquitoes of the Emulsions According to Example 1

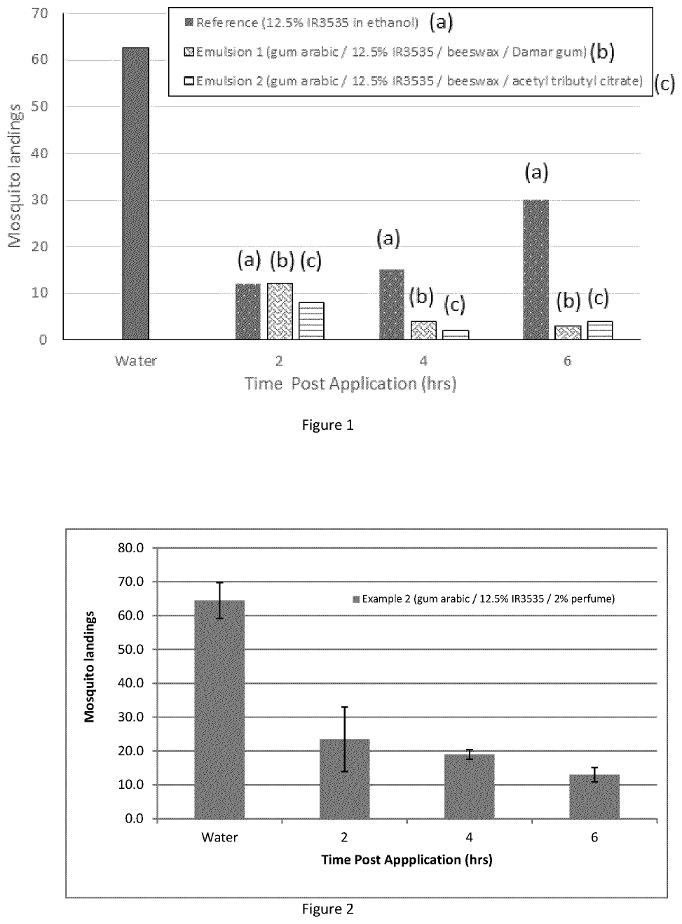

Controlling efficacy was tested against the yellow fever mosquito, Aedes aegypti Rockefeller strain. A. aegypti is a model organism for controlling tests and one of the recommended model organisms by the World Health Organization (WHO) as it is a very aggressive, anthropophilic mosquito species that shows generally low sensitivity to arthropod controlling compounds. Observations of controlling efficacy were made on host-seeking females of uniform age, 5 to 12 days post-eclosion from pupae. Tested hungry females had access to 10% sugar solution but were not blood-fed.
The testing protocol was adapted from the method described in Krober et al. (2010; doi: 10.2987/10-6044.1). A controlled amount (54) of stimulus was deposited in the center of a sandblasted glass plate (60 mm diam) and maintained at 34° C. The glass plate was then placed in a polycarbonate cage (200×260×180 mm) containing 30 A. aegypti unfed female mosquitoes. The number of landings of the mosquitoes as a function of time was monitored using a camera and allowed to evaluate the repulsive effect of the stimulus. Stimuli tested were water, ethanol or diluted insect repellent active as controls and samples according to example 1.
Results are shown in FIG. 1.
When only water is applied onto the glass surface, the average number of mosquitoes landing is about 63. The reference sample consisting of pure IR3535® diluted at 12.5% in ethanol reduces the number of landing from about 63 landings in the control test to about 12 landings 2 hrs after application (81% reduction), 15 after 4 hrs (−76%) and 30 after 6 hrs (−52%).
Emulsion 1 according to example 1 reduces the number of landings from about 63 landings in the control test to about 12 landings 2 hrs after application (81% reduction), 4 after 4 hrs (−93%) and 3 after 6 hrs (−95%). The result shows that the emulsion 1 according to the invention provides prolonged efficacy of the product and a good efficacy to repel mosquitoes during 6 hrs.
Emulsion 2 according to example 1 reduces the number of landings from about 63 landings in the control test to about 8 landings 2 hrs after application (87% reduction), 2 after 4 hrs (−97%) and 4 after 6 hrs (−93%). The result shows that the emulsion 2 according to the invention provides prolonged efficacy of the product and a good efficacy to repel mosquitoes during 6 hrs.

### Example 6

### Repulsive Effect on Mosquitoes of the Emulsion According to Example 2

The testing protocol was the same as in Example 5.
Results are shown in FIG. 2.
When only water is applied onto the glass surface, the average number of mosquitoes landing is about 60. Sample according to example 2 reduces the number of landings from about 60 landings with the control to about 23 landings 2 hrs after application (64% reduction), 19 after 4 hrs (−71%) and 13 after 6 hrs (−80%). The result shows that the sample according to the invention provides prolonged efficacy of the product and a good efficacy to repel mosquitoes during 4 hrs.

### Example 7

### Repulsive Effect on Mosquitoes of the Emulsion According to Example 3

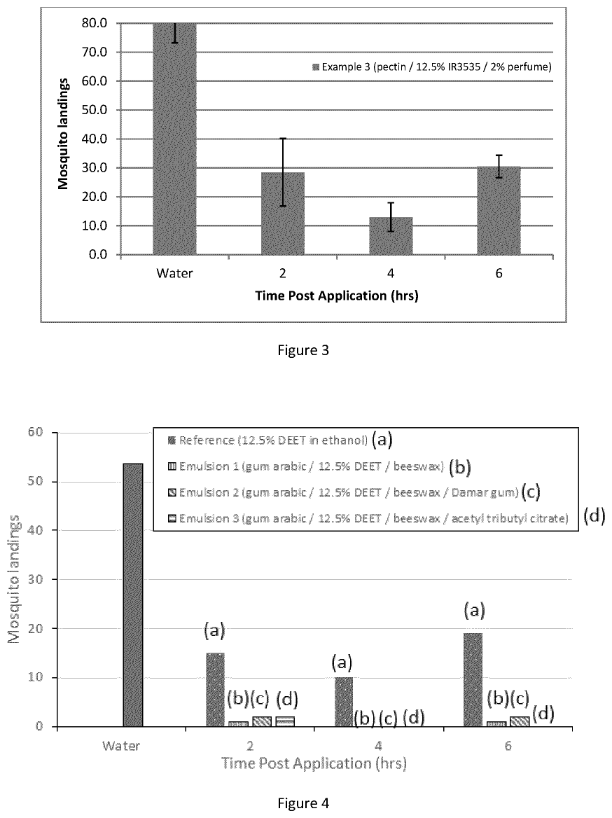

The testing protocol was the same as in Example 5.
Results are shown in FIG. 3.
When only water is applied onto the glass surface, the average number of mosquitoes landing is about 60. Sample according to example 3 reduces the number from about 80 landings with the control to about 28 landings 2 hrs after application (65% reduction), 13 after 4 hrs (−84%) and 30 after 6 hrs (−62%). The result shows that the sample according to the invention provides prolonged efficacy of the product and a good efficacy to repel mosquitoes during 4 hrs.

### Example 8

### Repulsive Effect on Mosquitoes of the Emulsions According to Example 4

The testing protocol was the same as in Example 5.
Results are shown in FIG. 4.
When only water is applied onto the glass surface, the average number of mosquitoes landing is about 54. The reference sample consisting of pure DEET diluted at 12.5% in ethanol reduces the number of landing from about 54 landings in the control test to about 15 landings 2 hrs after application (72% reduction), 10 after 4 hrs (−81%) and 19 after 6 hrs (−65%).
Emulsion 1 according to example 4 reduces the number of landings from about 54 landings in the control test to about 1 landing 2 hrs after application (98% reduction), 0 after 4 hrs (−100%) and 1 after 6 hrs (−98%). The result shows that the emulsion 1 according to the invention provides prolonged efficacy of the product and a good efficacy to repel mosquitoes during 6 hrs. Emulsion 2 according to example 4 reduces the number of landings from about 54 landings in the control test to about 2 landings 2 hrs after application (96% reduction), 0 after 4 hrs (−100%) and 2 after 6 hrs (−96%). The result shows that the emulsion 2 according to the invention provides prolonged efficacy of the product and a good efficacy to repel mosquitoes during 6 hrs.
Emulsion 3 according to example 4 reduces the number of landings from about 54 landings in the control test to about 2 landings 2 hrs after application (96% reduction), 0 after 4 hrs (−100%) and 0 after 6 hrs (−100%). The result shows that the emulsion 2 according to the invention provides prolonged efficacy of the product and a good efficacy to repel mosquitoes during 6 hrs.

### Example 9

### Emulsion of the Invention in the Form of a Gel

TABLE 5
Emulsion composition

| Ingredient | % |
|---|---|
| Demineralized water | 68.53 |
| Gum arabic¹⁾ | 8.20 |
| Xanthan gum²⁾ | 0.50 |
| Sodium citrate dibasic | 2.47 |
| sesquihydrate³⁾ | |
| Sodium citrate monobasic⁴⁾ | 1.15 |
| Sodium benzoate⁵⁾ | 0.07 |
| Potassium sorbate⁶⁾ | 0.07 |
| p-menthane-3,8-diol⁷⁾ | 12.50 |
| Fragrance⁸⁾ | 1.00 |
| Beeswax⁹⁾ | 1.50 |
| Tributyl-O-acetylcitrate¹⁰⁾ | 4.00 |

¹⁾Superstab ™ gum (origin: Nexira)
^{2, 3, 4, 9, 10)}Origin: Sigma Aldrich
^{5, 6)}Origin: Alfa Aesar
⁷⁾Citrepel ®75 (origin: Chemian Technology)
⁸⁾Origin: Firmenich

1. An oil-in-water emulsion comprising:
a dispersed oil phase comprising at least one solid lipid material and at least one liquid oil-soluble active ingredient, and
a continuous aqueous phase comprising a stabilizer,
wherein the oil-soluble active ingredient has a Log P>0.5.

2. The oil-in-water emulsion according to claim 1, wherein the amount of the oil-soluble active ingredient is comprised between 5% and 40% by weight, based on the total weight of the emulsion.

3. The oil-in-water emulsion according to claim 1, wherein the amount of solid lipid material is comprised between 1 to 20% by weight based on the total weight of the oil phase.

4. The oil-in-water emulsion according to claim 1, wherein the oil-soluble active ingredient comprises at least one arthropod control ingredient.

5. The oil-in-water emulsion according to claim 4, wherein the arthropod control ingredient is chosen from the group consisting of ethyl 3-(acetyl(butyl)amino)propanoate (IR3535®), N,N-diethyl-3-methylbenzamide (DEET), p-menthane-3,8-diol (PMD), Eucalyptus citriodora oil, Citronella spp. oil, sec-butyl 2-(2-hydroxyethyl)piperidine-1-carboxylate (picaridin), vanillin, Castor oil, Cedarwood oil, Cinnamon oil, Citronella, Citronella oil, Clove oil, Corn oil, Cornmint, Cornmint oil, Cottonseed oil, 4-Allyl-2-methoxyphenol (Eugenol), Garlic oil, (2E)-3,7-Dimethylocta-2,6-dien-1-ol (Geraniol), Geranium oil, Lemongrass oil, Linseed oil, Peppermint, Peppermint oil, 2-Phenylethyl propionate, Rosemary oil, Sesame oil, Soybean oil, Spearmint, Spearmint oil, Thyme oil, Mint, Mint oil, Pepper extract, Wintergreen oil, citronellal, Lavender oil, Lavandula hybrida extract, Lavandin oil, Lemon oil, Margosa extract, Mentha arvensis extract, Metofluthrin, Nonanoic acid, Pyrethrins and Pyrethroids, 2,3,4,5-bis(butyl-2-ene)tetrahydrofurfural (MGK Repellent 11), cineole, cinnamaldehyde, citral, citronellol, coumarin, dibutyl phthalate, diethyl phthalate, dimethyl anthranilate, dimethyl phthalate, ethyl anthranilate, ethyl vanillin, Eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxycitronellal, Lime oil, limonene, linalool, methyl anthranilate, myrcene, Neem oil, Sabinene, β-Caryophyllene, (1H-indol-2-yl)acetic acid, anethole, anise oil, basil oil, bay oil, camphor, ethyl salicylate, evergreen oils, pine oil, Tetramethrin, Allethrin, (RS)-a-cyano-3phenoxybenzyl-(1RS)-cis, Cypermethrin, Prallethrin, Acetamiprid, Azadirachtin, Bendiocarb, Bifenthrin, Chlorpyrifos, Deltamethrin, Diazinon, Dichlorvos, fipronil, imidacloprid, Malathion, Margosa extract, Nicotine, Permethrin, Rotenone, S-Methoprene, Spinosad (Spinosyn A), Spinosyn D, Transfluthrin, anisic alcohol, octahydrocoumarin, (+−)-2,5-dimethyl-2-indanmethanol, 4,4A,5,9B-tetrahydro-indeno[1,2-D]-1,3-dioxin, 2,4-dimethyl-4,4a,5,9b-tetrahydroindeno[1,2-d][1,3]dioxine, and mixtures thereof.

6. The oil-in-water emulsion according to claim 1, wherein the continuous phase comprises a hydrophilic active ingredient.

7. The oil-in-water emulsion according to claim 1, wherein the oil-soluble active ingredient comprises a mixture of an arthropod control agent and a perfume.

8. The oil-in-water emulsion according to claim 1, wherein the solid lipid material is chosen from the group consisting of a non-vegetable glyceride, vegetable glyceride, a non-vegetable wax, a vegetable wax, and mixtures thereof.

9. The oil-in-water emulsion according to claim 1, wherein the emulsion comprises an oil-miscible co-solvent, preferably and/or a water-miscible co-solvent.

10. The oil-in-water emulsion according to claim 1, wherein the stabilizer is chosen from the group consisting of Gum Arabic, Pectins, Casein, cyclodextrins, lecithins, soy protein, quillaja saponin, silica, calcium carbonate, zinc oxide, and mixtures thereof.

11. The oil-in-water emulsion according to claim 1, further comprising an additional component chosen from the group consisting of a weighting agent, a viscosifier, a gelling agent, and mixtures thereof.

12. Process for preparing an oil-in water emulsion, said process comprising the steps of:
(i) Dispersing an oil phase comprising at least one solid lipid material and at least one liquid oil-soluble active ingredient into a continuous aqueous phase comprising an stabilizer to obtain an oil-in-water emulsion, at a temperature above the melting point of the solid lipid material;
(ii) Cooling the emulsion thus obtained to a temperature below the melting point of the solid lipid material.

13. The process according to claim 12, wherein the melting point of the solid lipid material is comprised between 40° C. and 80° C.

14. A consumer product comprising the emulsion as defined in claim 1.

15. The consumer product according to claim 14, wherein it is in the form of a sprayable solution, or a gel/viscous lotion.

16. The oil-in-water emulsion according to claim 1, wherein the amount of the oil-soluble active ingredient is comprised between 10% and 30% by weight based on the total weight of the emulsion, and wherein the amount of solid lipid material is comprised between 5 to 15% by weight based on the total weight of the oil phase.

17. The oil-in-water emulsion according to claim 6, wherein the hydrophilic active ingredient is chosen from the group consisting of dried blood, lauryl sulfate, malic acid, potassium (2E,4E)-hexa-2,4-dienoate, putrescent whole egg solids, sodium chloride, sulfuric acid monododecyl ester, sodium salt, zinc, boric acid, citric acid, maltodextrin, silicium dioxide, and mixtures thereof.

18. The oil-in-water emulsion according to claim 7, wherein the arthropod repellent is used in an amount comprised between 5 and 40% based on the total weight of the emulsion, and wherein the perfume is used in an amount comprised between 0.1 to 5% by weight based on the total weight of the emulsion.

19. The oil-in-water emulsion according to claim 8, wherein the vegetable wax is chosen from the group consisting of beeswax, carnauba wax, palm stearin, jojoba wax and mixtures thereof.

20. The oil-in-water emulsion according to claim 9, wherein the oil-miscible co-solvent is chosen from the group consisting of benzyl benzoate, piperonil butoxide, coconut oil, and mixtures thereof and wherein the water-miscible co-solvent is chosen from the group consisting of ethanol, propanol, propylene glycol, hexylene glycol, dipropylene glycol, glycerol, di-isoproylidene glycol, butylene glycol (1,3-butanediol) and isopropanol, and mixtures thereof.
